(19) United States
(12) Patent Application Publication
(10) Pub. No.: US 2016/0250054 A1
(43) Pub. Date: Sep. 1, 2016

(54) System for Providing Intra-Oral Muscular Therapy, and Method of Providing Therapy for Intra-Oral Musculature for a Patient

(71) Applicant: Youhanna Al-Tawil, Knoxville, TN (US)

(72) Inventor: Youhanna Al-Tawil, Knoxville, TN (US)

(21) Appl. No.: 15/150,007

(22) Filed: May 9, 2016

Related U.S. Application Data
(63) Continuation-in-part of application No. 14/231,626, filed on Mar. 31, 2014.
Application No. 14/231,626 is a continuation-in-part of application No. 13/358,216, filed on Jan. 25, 2012, now Pat. No. 8,961,437.
Application No. 13/358,216 is a continuation of application No. 13/092,234, filed on Apr. 22, 2011, now Pat. No. 8,579,766.
Application No. 13/092,234 is a continuation-in-part of application No. 12/782,356, filed on May 18, 2010, now Pat. No. 8,047,964.
Application No. 12/782,356 is a continuation-in-part of application No. 12/556,237, filed on Sep. 9, 2009, now Pat. No. 7,942,782.
(60) Provisional application No. 61/096,408, filed on Sep. 12, 2008.

(51) Int. Cl. A61F 4/00; G06F 3/01

(52) U.S. Cl. CPC A61F 4/00; G06F 3/015

(57) ABSTRACT

An intra-oral system for facilitating therapeutic treatment of a patient's intra-oral musculature is provided. Also provided are methods of therapeutic treatment for intra-oral musculature. The system and methods employ a mouth piece having two or more pressure cells, such as fluid pressure cells. A patient or user applies pressure to the cells using their tongue. Wireless signals are sent to an external micro-processor, with the signals being indicative of location of pressure, degree of pressure, or both as applied to the pressure cells. A therapist may analyze pressure data as part of a therapeutic regimen.

### CROSS REFERENCE TO RELATED APPLICATIONS

This application is a continuation-in-part of non-provisional patent application U.S. Ser. No. 14/231,626 filed Mar. 31, 2014. That application published as U.S. Publ. No. 2015/0277759 and is entitled “Mouth Piece for Manipulating an External Object, and Method for Moving a Cursor on Display.”

U.S. Ser. No. 14/231,626 was filed as a continuation-in-part of U.S. Ser. No. 13/358,216, filed Jan. 25, 2012 which is entitled “Mouth Piece for Manipulating an External Object, and Method for Moving a Cursor on Display.” The '626 application issued in 2015 as U.S. Pat. No. 8,961,437 and is entitled “Mouth Guard for Detecting and Monitoring Bite Pressures.”

U.S. Ser. No. 13/358,216 was filed as a continuation-in-part of U.S. Ser. No. 13/092,234, filed Apr. 22, 2011. That application was entitled “Head Set for Lingual Manipulation of an Object, and Method for Moving a Cursor on a Display,” and issued in 2013 as U.S. Pat. No. 8,579,766.

U.S. Ser. No. 13/092,234 was filed as a continuation-in-part of U.S. Ser. No. 12/782,356, filed May 18, 2010. That application was entitled “Methods and Systems for Lingual Movement to Manipulate an Object,” and issued in 2011 as U.S. Pat. No. 8,047,964. U.S. Ser. No. 13/092,234 was also filed as a continuation-in-part of U.S. Ser. No. 12/556,237, filed Sep. 9, 2009. That application was also entitled “Methods and Systems for Lingual Movement to Manipulate an Object,” and issued in 2011 as U.S. Pat. No. 7,942,782.

U.S. Ser. No. 12/556,237 claimed the benefit of U.S. Prov. Appl. No. 61/096,408 filed Sep. 12, 2008. These prior applications are incorporated herein by reference in their entireties.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### BACKGROUND OF THE INVENTION

1. Field of the Invention

The present invention relates to therapeutic devices. More specifically, the present invention relates to methods and systems for helping patients improve mastication and deglutition. The present invention also has application to the movement of an object on a visual display using lingual manipulation in order to move a mechanical object, to activate an electrical apparatus, or to type on a digital keyboard. The system is particularly beneficial for assisting an individual who is impaired in the use of his upper extremities.

2. Technology in the Field of the Invention

Some individuals have limited use of their upper extremities. Such individuals may, for example, have suffered a stroke. The term “stroke” is a lay term that refers to a condition wherein the blood supply to an area of the brain is temporarily cut off. When blood fails to get through to parts of the brain, the oxygen supply to those areas is cut off. Without oxygen, brain cells die. The longer the brain is without blood, the more severe the damage will be. Where the portion of the brain that controls movement of the upper extremities is damaged, the individual may be left in a state of paralysis.

Individuals may also lose function of their upper extremities as a result of an injury. Such injuries may occur due to a car accident, a diving accident, a fall, or other trauma. In these instances, the individual's cervical spine and nerves may be injured, producing partial or complete paralysis of the hands or arms.

In addition to these events, some individuals may develop paralysis as a result of a medical condition. Examples of such conditions include amyotrophic lateral sclerosis (ALS), hypokalemic periodic paralysis, or other diseases. Finally, some individuals may completely lose all or a portion of both arms due to an explosion or accident incident to military duty or first-responder service.

When any of these conditions of paralysis or injury occur, the individual is left without the ability to move an object using his or her arms. Thus, the individual cannot turn off a light, adjust a bed, change a channel, send a text message, or perform countless other activities that most people take for granted.

Assistive devices have been presented for disabled persons. U.S. Pat. No. 6,833,786 presents a pneumatic de-multiplexer that utilizes a “sip-and-puff” tube for manipulating an appliance. The sip-and-puff technology allows a user to selectively inhale and exhale to cause movement of a wheelchair or to operate another appliance. However, sip-and-puff technology would be extremely cumbersome for typing a message on a display or for navigating web-based applications on a micro-computer.

U.S. Pat. No. 7,071,844 describes a wireless, tongue-operated device for controlling electronic systems. The device is said to utilize a single electrical sensor embedded in an oral sensor-mounting device, such as a dental retainer or a mouth-guard. The sensor is said to generate electrical signals to an interface, which then processes the signals into control signals. The '844 patent fails to identify a source, name, or model number for the depicted electrical sensor. Further, the '844 patent fails to describe how the sensor would be powered (neither a power wire nor a cable is shown or mentioned), and fails to explain where one would obtain or how one would design a small, intra-oral electrical sensor offering multiple detected regions for computing movement of a user's tongue.

U.S. Pat. No. 6,702,765 provides a mouth piece having electrical sensors placed on a “hard palate” member. The hard palate member is sometimes referred to as a “sensor support strip.” Electrically conductive wires extend from the support strip to an “external annunciator unit.” The external unit is used to measure or calibrate pressure as applied by the tongue of a user. The external unit includes a micro-controller with onboard memory where target pressures are recorded. However, the mouth piece does not control an external device in real time, nor does it send a wireless signal to the micro-controller.

Therefore, a need exists for an apparatus, in one embodiment, that will allow an individual having limited use of their upper extremities to move an object using lingual manipulation. Further, a need exists for a head set having a connected mouth piece that allows an individual to move a cursor on a display using lingual manipulation. Additionally, a need exists for a therapeutic system that enables a patient to improve intra-oral muscle strength and dexterity through the application of pressure on pressure cells, where pressure signals are converted into electrical signals that are transmitted via a wireless protocol to an external micro-processor.

### BRIEF SUMMARY OF THE INVENTION

An intra-oral system is first provided herein. The system is beneficial for assisting an individual who is significantly impaired in the use of his or her upper extremities. The system enables such an individual to manipulate an object. The object may be a mechanical device such as a door or a bed. Alternatively, the object may be an appliance, wherein “moving” the appliance means turning it on, off, up or down. Alternatively still, the object may be a cursor on a digital display that facilitates the typing of alphanumeric characters on a virtual keyboard.

In another aspect, the intra-oral system is a therapeutic device that has efficacy for patients who have limited use of their tongue and intra-oral musculature. For example, as noted in the parent applications incorporated herein, the patient may have problems with chewing (mastication) or swallowing (deglutition), or may have suffered a stroke (paresis).

In any embodiment, the intra-oral system includes a pliable mouth piece. In one aspect, the mouth piece is an inter-dental device dimensioned to reside around the maxillary (or upper) teeth of a user. The mouth piece comprises opposing left and right sides, and an arcuate portion intermediate the left and right sides to form a generally horseshoe-shaped member.

The system also includes a palate member. The palate member resides between the left and right sides of the mouth piece, and is part of the mouth piece.

The mouth piece, including the palate member, may be fabricated from an elastomeric material. Examples of elastomeric materials include polyisoprene rubber, chloroprene rubber, silicone rubber, neoprene, styrene butadiene rubber, acrylonitrile butadiene rubber, ethylene propylene diene methylene, polyvinylchloride, polyethylene, polyurethane, urethane-coated nylon, ethyl vinyl acetate, and combinations thereof. Alternatively, the mouth piece may be fabricated from a plasticized acrylic gel. Alternatively still, the mouth piece may be fabricated from a moldable thermoplastic material that softens when immersed in boiling water, and then sets when cooled. The thermoplastic material is also known as a “boil-and-bite” mouth guard. In any event, the U-shaped body is dimensioned to fit inside a user's mouth and over the maxillary teeth.

The system further includes at least two pressure cells. Preferably, the cells are fluid pressure cells. The cells are affixed to a surface of the palate member and are configured to respond to pressure applied by the tongue of the user. Thus, using their tongue, the patient or other user applies pressure to the cells to manipulate the external object or to generate wireless signals for therapeutic analysis.

In one embodiment, the mouth piece comprises at least four directional cells. The directional cells may optionally be disposed radially around a center point. The center point defines a separate cell, referred to as an “enter” cell. In one aspect, the enter cell is disposed to the side or is otherwise eccentric to the directional cells.

Each of the cells preferably contains a fluid. The fluid may be air or some other inert gas. In this instance, the cells may be referred to as air cells. Alternatively or in addition, the fluid may be water or other non-toxic liquid. In this case, the cells are fluid cells. For ease of reference, the cells in either embodiment may be described herein as fluid cells or simply as cells.

A plurality of transducers is also provided. Each transducer is in fluidic communication with a respective pressure cell. The transducers convert changes in pressure within the respective cells to corresponding electrical signals. Such electrical signals may be, for example, voltage signals, current signals, or resistive changes. The transducers are preferably in the nature of pressure sensors.

The system further includes a processor. The processor serves to process the electrical signals. The processor may include an analog-to-digital converter, meaning that electrical signals from the pressure sensors are converted into digital values. The converted electrical signals, such as voltage signals, may be modulated to move a cursor on a display. The cursor may move one incremental step in a pre-designated direction. Repeatedly applying pressure to a directional cell will cause the cursor to move in that designated direction across the display.

The processor may be in operational electrical communication with a motor. For example, the processor may send instructions through a wire or a transmitter that causes the motor to move an object such as a bed, a door or a wheelchair. Alternatively, the processor may be in operationally electrical communication with a switch. The switch changes the electrical state of an appliance such as a light fixture, a television, or a thermostat.

The processor is preferably in communication with a display. In this embodiment, the display provides a visual platform for the movement of a cursor in accordance with the pressure applied to the directional cells. The cursor is manipulated by application of lingual pressure on the directional cells to provide two-dimensional, or x-y movement.

The cursor is moved to a designated key or symbol. The key or symbol is then “clicked” in order to activate a motor on a device or to change the state of an electrical appliance or to type a letter or text message. Clicking is done by applying pressure to the enter cell. Thus, the center point cell serves as a digital “Enter” button.

In one aspect, the mouth piece and connected palate member are affixed to a head set. The head set is configured to be worn on a head of the user. Alternatively, the mouth piece may be supported by a support member near the user, or may be free standing.

Where a head set is used, the head set will include a head piece, and an articulating arm extending from the head piece and having a distal end supporting the mouth piece. In one preferred embodiment, the processor and the transducers are mechanically supported by the head set.

In one aspect, the transducers and the processor are part of a first processing unit. The first processing unit comprises a transmitter. The intra-oral system then further comprises a second processing unit, or micro-processor, associated with a display. The external object is a cursor on the display. The transmitter is configured to communicate with the second processing unit via wireless signals to move the cursor. Alternatively, the micro-processor is associated with operational software that enables a therapist to analyze pressure data received in the form of the wireless signals.

A method of controlling an external device using lingual manipulation of one or more pressure cells is also provided. In one aspect, the method first comprises placing a mouth piece at least partially within a user's mouth. The mouth piece has at least two fluid pressure cells.

The method also includes applying pressure to a pressure cell of the at least two fluid pressure cells. This is done by the user through lingual manipulation. In response to the pressure applied to the pressure cell of the at least two pressure cells, the method includes converting pressure values into electrical signals.

The method further includes processing the electrical signals to correlate pressure applied by lingual manipulation on the pressure cells of the mouth piece with (i) locations on the mouth piece, (ii) degrees of pressure applied to the pressure cells on the mouth piece, or (iii) both. The electrical signals are then transmitted as pressure data in the form of wireless signals from the mouth piece to a micro-processor. In response to the receiving of wireless signals, the micro-processor moves an object. Alternatively, the micro-processor records pressure data to be used for analysis by a therapist.

The mouth piece is configured in accordance with any of the embodiments described above. For example, the mouth piece may comprise a pliable palate member dimensioned to be received against a palate of the user, and each of the at least two pressure cells may be located on a surface of the palate member. Preferably, the mouth piece further comprises transducers associated respectively with each of the two or more pressure cells.

In one aspect, the mouth piece is affixed to a head set configured to be worn on a head of the user. The method then further comprises placing the head set onto the head of the user, and inserting the mouth piece into the user's mouth.

In one aspect, transmitting the electrical signals as wireless signals from the pressure cells comprises transmitting the electrical signals as wireless signals from a transceiver in electrical communication with the transducers. The wireless signals may be used to select characters on a keyboard in order to compose a textual message. Alternatively, the wireless signals represent pressure data that is analyzed to determine a patient's (i) lingual strength, (ii) lingual dexterity, or (iii) both as part of a therapeutic treatment for (i) deglutition, (ii) lingual strength, or (iii) both.

A method of providing therapy for intra-oral musculature is also provided herein. In one embodiment, the method first comprises placing a mouth piece at least partially within a patient's mouth, with the mouth piece having at least two pressure cells. Preferably, each of the at least two cells is located on a surface of the palate member.

The method also includes applying pressure to a pressure cell of the at least two pressure cells through lingual manipulation. In response to pressure applied to the pressure cell of the at least two pressure cells through lingual manipulation, the method includes converting pressure values into electrical signals.

The method additionally includes processing the electrical signals to correlate pressure applied by lingual manipulation on each of the pressure cells of the mouth piece with (i) locations on the mouth piece, (ii) degrees of pressure applied to the pressure cells on the mouth piece, or (iii) both. The method then includes transmitting the electrical signals as wireless signals from the mouth piece to a micro-processor.

The method then further includes analyzing the wireless signals to determine a patient's (i) lingual strength, (ii) lingual dexterity, or (iii) both as part of a therapeutic treatment, wherein the treatment is for (i) deglutition, (ii) lingual strength, or (iii) both.

In one aspect, the method also includes placing the plurality of tubes in fluid communication with the corresponding transducers. In one aspect, the transducers are part of a first processing unit. The first processing unit further comprises a plurality of jumper tubes in fluid communication with respective transducers. The intra-oral system further comprises a ribbon cable for receiving respective jumper tubes in channels at a proximal end, and for receiving respective fluid tubes in channels at a distal end. In this way, the fluid cells and the transducers are placed in fluid communication.

Preferably, the first processing unit further comprises a transmitter. The intra-oral system then further comprises a second processing unit associated with a display. The display may be used by a therapist to review and evaluate pressure values generated by a patient's tongue.

In one aspect, the transmitter is an infrared controller. In another aspect, the transmitter is a first transceiver that uses Bluetooth, Wi-Fi, or other wireless technology to send command signals that correspond to the pressure readings from the cells. In one aspect, the transmitter delivers the commands to a transceiver, which is in electrical communication with a second processor. The second processor causes a cursor to move across the display, with the display having alphanumeric and/or other symbols. Thus, the first processor communicates wirelessly with the display via the second processor. The second processor and display may be part of a portable digital assistant such as the iPhone® or the iPad® of Apple, Inc. of Cupertino, Calif.

In one aspect, the processor is in electrical communication with a motor for moving the external object, wherein the object is a bed, a wheelchair, or a door. In another aspect, the processor is in electrical communication with a switch for changing an electrical state of an appliance, wherein the switch controls a light fixture, a television, or a thermostat.

In one embodiment, the method further comprises selecting a symbol on the display to actuate a mechanical device or an electrical appliance. The symbol on the display may comprise a picture, one or more alphanumeric characters, an arrow, or a geometric figure. Alternatively or in addition, one or more symbols on the display comprise a keyboard such that the user may select a series of characters on the keyboard using their tongue to composing a textual message. The method may then include selecting a “send” symbol on the display that, when selected by the user, the textual message is sent through a wireless communications system.

Finally, a method of typing characters on a virtual keyboard using lingual musculature is provided herein. In one embodiment, such method includes placing an elastomeric mouth piece in a user's mouth. The mouth piece is generally constructed as described for the intra-oral system above. The mouth piece includes a palate member residing between and supported by left and right sides of the mouth piece. The palate member supports a plurality of fluid-containing cells on an under-surface.

The method also includes placing the cells in fluid communication with corresponding transducers. This is done by connecting tubes between the transducers and the cells. Thus, a proximal end of the tubes is connected to the transducers, while a distal end is in fluid communication with the respective cells.

Preferably, the plurality of cells includes four directional cells and an enter cell. The directional cells are used to move the cursor along an x-y plane, while the enter cell is used to execute a function, such as selecting a letter on the keyboard.

The method next includes applying pressure to the directional cells in order to move a cursor on a display. Pressure is applied by the user through his or her tongue. The method then includes moving the cursor to selected symbols on the virtual keyboard on the display, and then actuating the selected symbols, thereby composing a textual message. Actuating the selected symbols is done by applying lingual pressure to the enter cell.

In operation, applying pressure to the cells causes changes in fluid pressure within the plurality of tubes. These changes are sensed by each of the plurality of transducers and converted to an electrical signal. The transducers are in operative electrical communication with a processor which processes the electrical signals in order to move the cursor. The processor is in operative electrical communication with the display for causing the cursor to move across the virtual keyboard.

The method may next include sending the textual message through a wireless communications system. Preferably, the processor transmits operational commands to a second processor through a transmitter. The second processor is associated with a display having the virtual keyboard. The second processor and display are a tablet or a so-called smart phone.

### BRIEF DESCRIPTION OF THE DRAWINGS

So that the manner in which the present invention can be better understood, certain illustrations, charts and/or flow charts are appended hereto. It is to be noted, however, that the drawings illustrate only selected embodiments of the inventions and are therefore not to be considered limiting of scope, for the inventions may admit to other equally effective embodiments and applications.

FIG. 1 is a perspective view of an intra-oral system according to the present invention, in one embodiment. A head set is seen as part of the illustrative system, with the head set having a connected mouth piece.

FIG. 2A is a perspective view of the exemplary head set of FIG. 1.

FIG. 2B is a cross-sectional view of a tube bundle from the head set of FIG. 2A.

FIG. 2C is another perspective view of a head set for an intra-oral system, in an alternate embodiment.

FIG. 3A is a perspective view of the mouth piece from the intra-oral system of FIG. 1, in one embodiment. The view is seen from a front of the mouth piece.

FIG. 3B is a bottom view of the mouth piece from the system of FIG. 1. Here, various fluid cells are seen disposed on a bottom of a palate member of the mouth piece.

FIG. 3C is a rear view of the mouth piece from the system of FIG. 1. A portion of several of the fluid cells are shown along with an illustrative manifold for receiving respective fluid tubes.

FIG. 3D is another perspective view of the mouth piece of FIG. 2A. Here, a rear perspective view is provided.

FIG. 4A is a schematic view of the fluid tubes as may be used in the intra-oral system of FIG. 1 extending from the end of a ribbon cable. Respective fluid tubes are seen in fluid communication with four illustrative “directional” cells and a centrally-located “enter” cell. The mouth piece has been removed for illustrative purposes.

FIG. 4B is a perspective view of the ribbon cable used for protecting the individual tubes as they extend from the manifold of the mouth piece to an electronics box.

FIG. 5A is a perspective view of the mouth piece of FIG. 2A juxtaposed with an integrated electronics box. The housing is removed from the electronics box, revealing components for processing pressure signals. In this arrangement, no separate head set is needed.

FIG. 5B is a perspective view of the mouth piece of FIG. 2A, again juxtaposed with the integrated electronics box. Here, the housing has been placed onto the processor, with the processor exploded away from a tube-connector.

FIGS. 6A through 6C present various arrangements for displays from the system of FIG. 1.

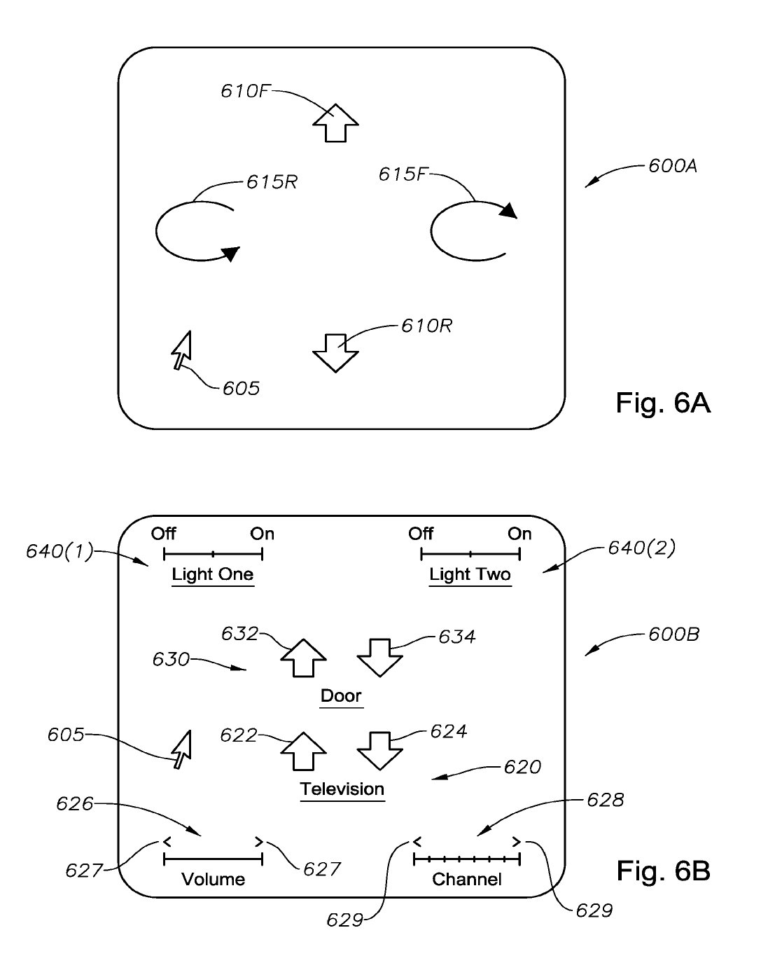

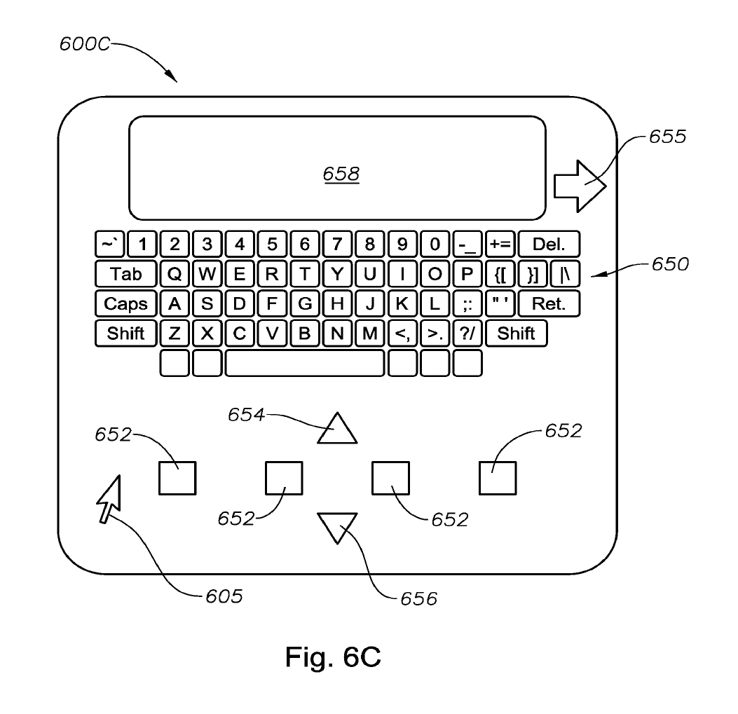

In FIG. 6A, the display shows a cursor that may be moved on a display. The cursor is moved through lingual manipulation in order to operate a wheelchair or other mechanical device.

In FIG. 6B, the display also shows a cursor that may be moved on a display. Here, the cursor is moved through lingual manipulation in order to change the status of an electrical appliance.

In FIG. 6C, the display again shows a cursor that may be moved on a display. Here, the cursor is moved through lingual manipulation in order to digitally “press” or “click” on keys from a virtual keyboard.

### DETAILED DESCRIPTION OF CERTAIN EMBODIMENTS

### Definitions

As used herein, the term “cursor” means any indicator of a position on a computer screen or display. The cursor may be, for example, a flashing bar, an underline, an arrow or other symbol.

### Description of Specific Embodiments

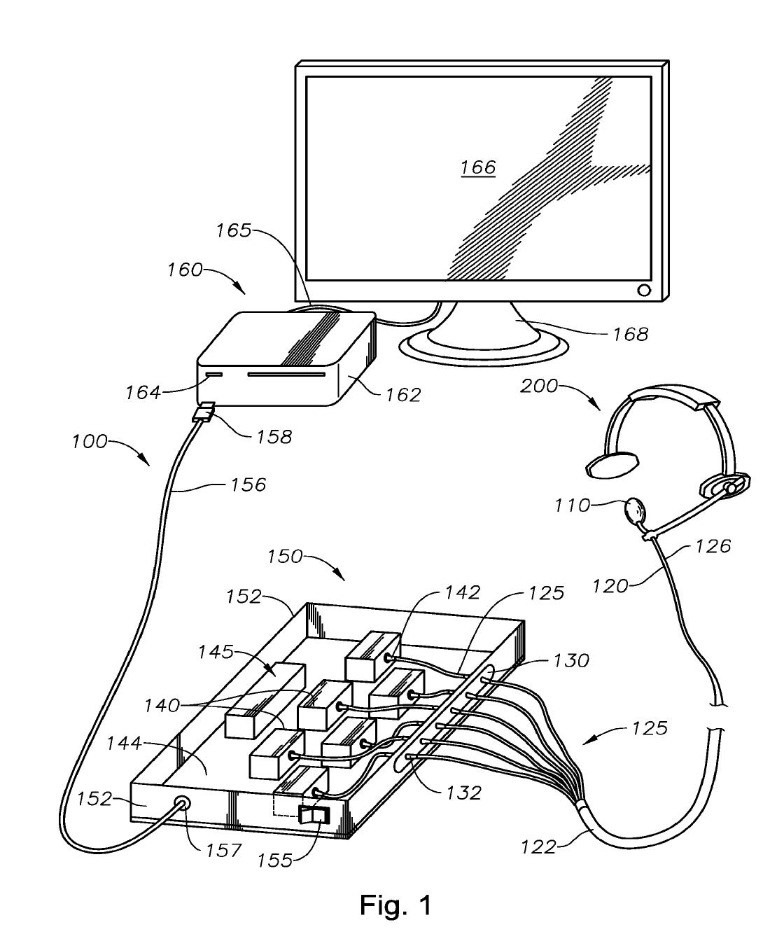

FIG. 1 is a perspective view of an intra-oral system 100 according to the present invention, in one embodiment. Various components of the system 100 are shown. The components first include a head set 200, shown in one embodiment. The head set 200 is designed and configured to be worn on the head (not shown) of a user. The user is preferably an individual who has lost function of at least their hands and, possibly, additional portions of their upper extremities. However, the head set 200 may be worn by any individual.

The system 100 includes various other components such as a mouth piece 110, a plurality of tubes 125, a manifold 130, a plurality of corresponding transducers 140, a processor 145, an operational box 150 (or housing), a computer 160 (representative of a second processor), and a display 166.

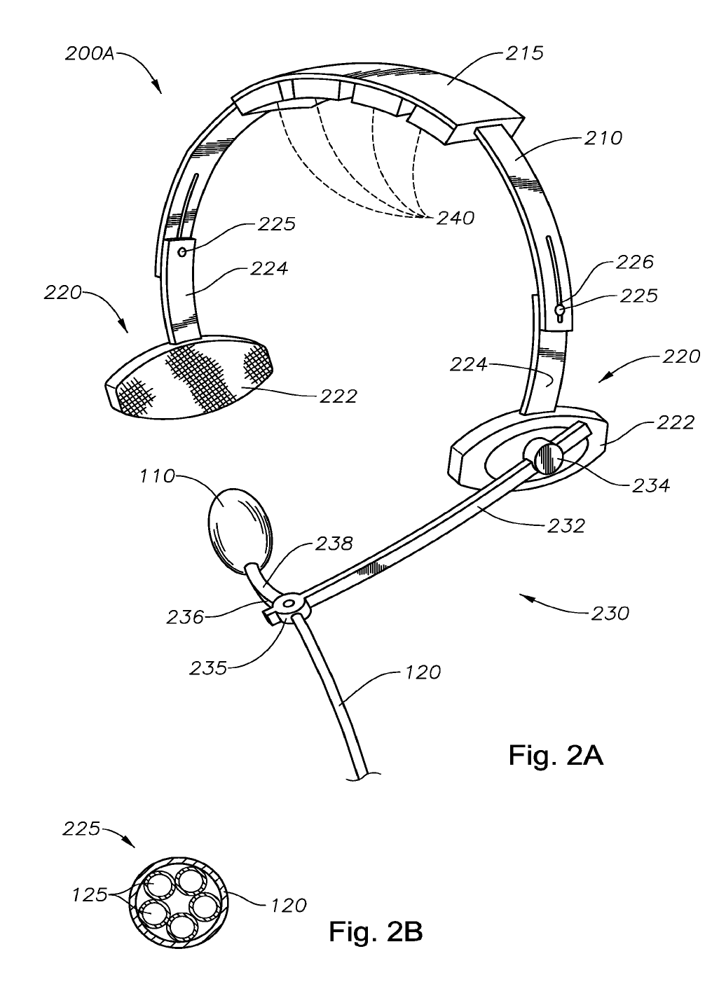

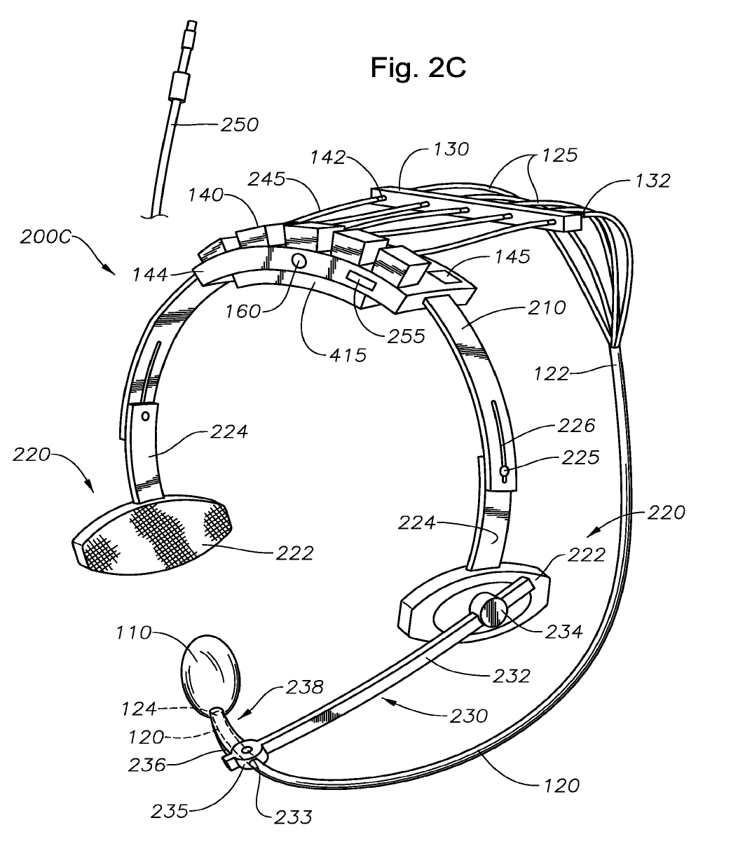

FIG. 2A provides an enlarged perspective view of the head set 200. Here, the head set is shown individually, and is identified by reference number 200A. The head set 200A first includes a support member 210. The support member 210 defines an arcuate or arched member configured to rest on the crown of an individual user's head. The support member 210 optionally includes a central cushioning member 215.

The head set 200A also includes opposing head rests 220. In the arrangement of FIG. 2, the head rests 220 each include pads 222 and supporting bars 224. The supporting bars 224 include pins 225. The pins 225 slidably move through slots 226 in the support member 210. In this way, one or both of the head rests 220 is adjustable relative to the head set 200A.

The head set 200A also includes an articulating arm 230. In the arrangement of FIG. 2A, the articulating arm 230 has a first arm portion 232 and a second arm portion 238. The first arm portion 232 has a proximal end 234 slidably and pivotally connected to one of the pads 222. The first arm portion 232 also has a pivot point 235 opposite the proximal end 234.

The second arm portion 238 pivots from the pivot point 235 of the first arm portion 232. Opposite the pivot point 235, the second arm portion 236 has a distal end 238. The mouth piece 110 is connected to the articulating arm 230 at the distal end 238.

It is understood that the configuration of the head set 200A and its articulating arm 230 are merely illustrative. Other designs and arrangements may be employed. What is important is that the head set 200A be designed to allow the mouth piece 110 to reach the mouth of the user.

The mouth piece 110 is configured to be selectively inserted into an individual's mouth (not shown). As noted, the individual is preferably a person who has limited use of their upper extremities. However, the individual may also be a patient who is in need of therapy to develop the intra-oral musculature. Such a patient may be, for example, a stroke victim or the victim of a head or neck injury. Alternatively, such a patient may be a child who suffers from congenital limitations in chewing and/or swallowing food.

The mouth piece 110 is preferably fabricated from an elastomeric material. Suitable materials may include polyisoprene rubber, chloroprene rubber, neoprene rubber, styrene butadiene rubber, and acrylonitrile butadiene rubber. Additional suitable examples include silicone, ethylene propylene diene methylene, polyvinylchloride, polyethylene, polyurethane, urethane-coated nylon, and ethyl vinyl acetate. Combinations of these materials may also be employed.

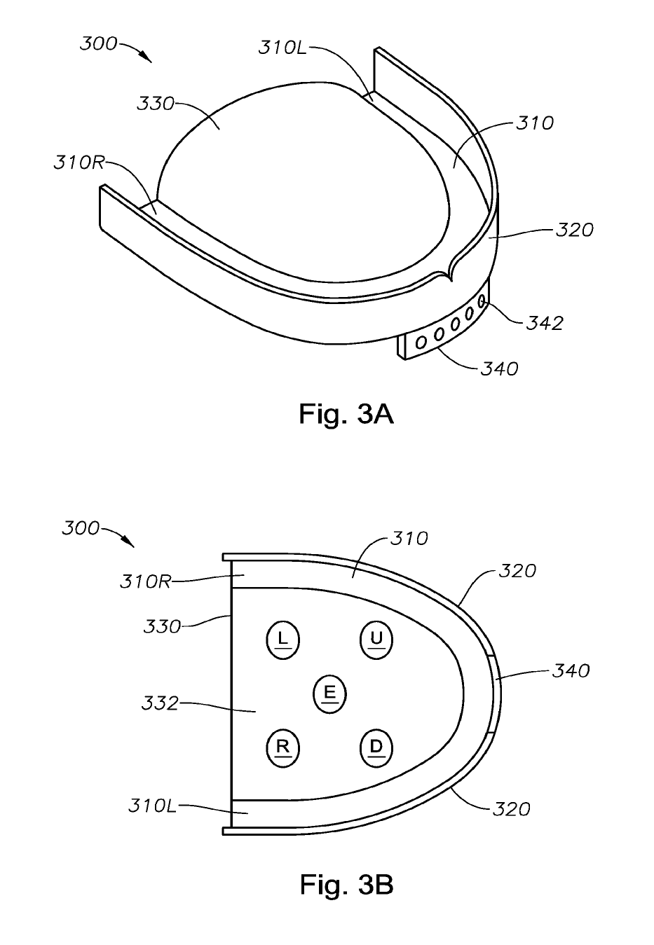

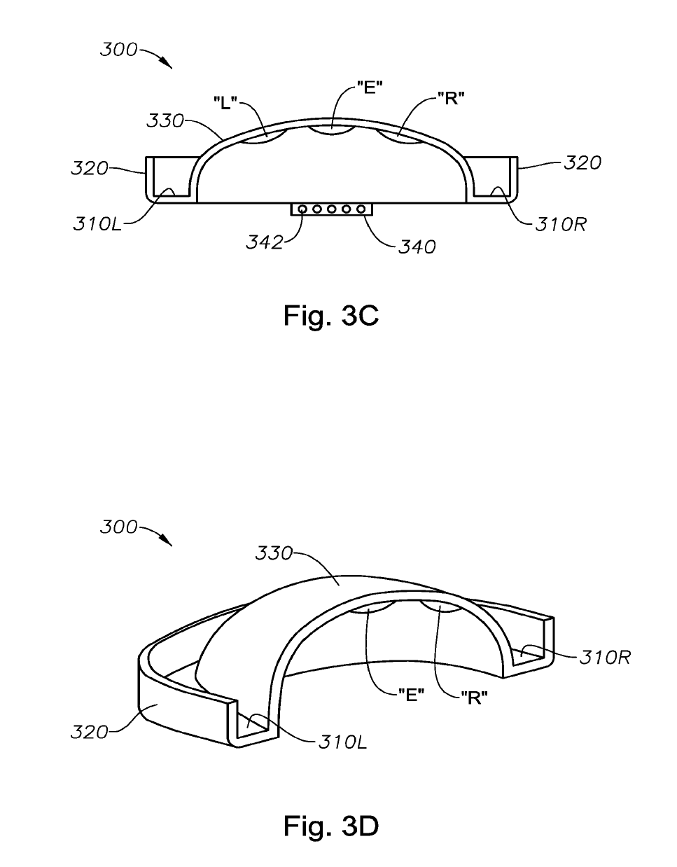

It is noted that the mouth piece 110 shown in FIGS. 1 and 2A is somewhat schematic. More precise views of the mouth piece 110 are provided in FIGS. 3A through 3D. FIG. 3A is a perspective view of a mouth piece 300 as may be used with the head set 200A, in one embodiment.

Referring to FIGS. 3A through 3D together, it is first seen that the mouth piece 300 has a circumferential channel 310. The channel 310 is designed to receive the teeth (not shown) of a user. The mouth piece 300 also has U-shaped or horseshoe-shaped wall 320. The wall 320 is dimensioned to fit around the maxillary teeth of the user.

The channel 310 of the mouth piece 300 has left 310L and right 310R sides. Intermediate the left 310L and right 310R sides is a palate member 330. The palate member 330 is dimensioned to generally fit against the roof of the user's mouth. In addition, the palate member 330 is configured to support a series of fluid cells on an under-surface 332.

In FIG. 3A, the under-surface of the palate member 330 and the cells are not seen. However, FIG. 3B provides a bottom view of the mouth piece 300. Here, the under-surface 332 of the palate member 330 is seen. In addition, a plurality of fluid cells are shown. The fluid cells are identified as “L,” “R,” “U,” “D,” and “E.” The fluid cells may be identified as follows:

Pressure applied to cell “L” causes a cursor on a display to move to the left;

Pressure applied to cell “R” causes the cursor to move to the right;

Pressure applied to cell “U” causes the cursor on the display to move up;

Pressure applied to cell “D” causes the curser to move down; and

Pressure applied to cell “E” causes an action to occur.

Thus, the mouth piece serves as a “mouth mouse.”

Each of the cells “L,” “R,” “U,” “D,” “E” is filled with a fluid. The fluid may be a compressible fluid, or gas. The compressible fluid may be air or another inert gas. The compressible fluid may comprise oxygen, carbon dioxide, nitrogen, or combinations thereof. Alternatively, the fluid may be a substantially non-compressible fluid, such as water or other non-toxic liquid. A combination of compressible and non-compressible fluids may also be employed. In any instance, fabrication of the intra-oral system 100 will typically involve establishing a baseline pressure between the cells “L,” “R,” “U,” “D,” “E” and electronics. This establishes a more accurate conversion of pressure changes to electrical signals by the transducers 140.

Each cell “L,” “R,” “U,” “D,” “E” holds a volume of fluid. Preferably, the fluid is held at ambient pressure. Alternatively, the fluid in the cells “L,” “R,” “U,” “D,” “E” is pre-loaded at a higher pressure such as between about 15 psi and 25 psi. In this way, the mouth piece 110 is at least nominally resistive to pressure placed by the patient using his or her tongue.

The cells “L,” “R,” “U,” “D,” “E” are affixed to the under-surface 332 of palate member 330 independently. Preferably, the cells do not share walls, but have their own walls. The walls are sealed to the under-surface 332 through heat sealing, RF sealing, or other mechanisms known in the art of plastic injection molding or other molding techniques. Alternatively, the cells may be connected using a non-toxic adhesive. Alternatively, the cells are fabricated integral to the palate member 330 through an injection molding process.

Additional views of the mouth piece 300 are provided. FIG. 3C is a rear view of the mouth piece 300. A portion of the fluid cells “L,” “E,” and “R” are shown. Of interest, the relative dimensions of the channel 310 and the surrounding wall 320 are seen. Of course, the mouth piece 300 may be configured in different sizes. The size will primarily be dictated by the size of the individual user's mouth. FIG. 3D is another perspective view of the mouth piece 300 of FIG. 3A. Here, a rear perspective view is provided. The concave shape of the palate member 330 is readily seen.

The mouth piece 300 may include an optional manifold 340. The manifold 340 is disposed immediately below the wall 320 at a front of the mouth piece 300. The manifold 340 includes a plurality of through-openings 342. The through-openings 342 are dimensioned to receive respective tubes, such as tubes 125 from FIG. 1. The tubes 125, in turn, extend to the fluid cells “L,” “R,” “U,” “D,” “E.”

Referring back to FIG. 1, the system 100 also includes a plurality of tubes 125. A tube 125 is provided to correspond to each cell “L,” “R,” “U,” “D,” “E.” The tubes 125 are sealingly received within the walls of the respective mouth piece cells “L,” “R,” “U,” “D,” “E.”

In the arrangement of FIG. 1, the tubes 125 are optionally bundled as they exit the mouth piece 110. That means that the tubes 125 are held together externally by a tubular sheath 120. The tubular sheath 120 is also seen in FIG. 2A extending from the pivot point 235 of the head set 200A. Of course, the tubular sheath 120 need not travel through the pivot point 235 of the head set 200.

FIG. 2B is a cross-sectional view of a tube bundle 225 from the head set of FIG. 2A, in one embodiment. In the arrangement of FIG. 2B, the tube bundle 225 includes the tubular sheath 120. The tubular sheath 120 helps to protect the tubes 125 and keeps them from getting punctured or tangled. Six illustrative tubes 125 are seen within the tubular sheath 120. Each tube 125 defines a channel through which fluid passes. It is understood that any number of tubes 125 and corresponding cells may be used in the system 100.

Referring again to FIG. 1, the system 100 also includes a plurality of transducers 140. The transducers 140 are in the nature of pressure sensors. The transducers 140 may be, for example, ASDX pressure sensors made by the Sensing and Control Division of Honeywell in Golden Valley, Minn. The ASDX series of pressure sensors utilize a small internal diaphragm for sensing fine variations in pressure. Different sensors are offered in the series for sensing within different pressure ranges. Such ranges include 0 to 1 psi, 0 to 5 psi, 0 to 15 psi, and 0 to 30 psi. The ASDX sensors offer a high level output (5.0 Vdc span) that is fully calibrated and temperature compensated with on-board Application Specific Integrated Circuitry (ASIC).

The transducers 140 are housed within the operational box 150. The box 150 has walls 152 and a top (not shown). The operational box 150 will include an electrical circuit board 144 that places the transducers 140 in electrical communication with the processor 145 as well as with a power supply. A power switch for the operational box 150 is seen at 155.

The transducers 140 are in fluid communication with respective cells “L,” “R,” “U,” “D,” and “E.” This is done by means of the tubes 125. A proximal end of each tube 125 is connected to a transducer 140 at a connection point 142, while a distal end of each tube 125 is connected to a respective cell, preferably through the through-openings 342 in the manifold 340.

Each of the tubes 125 may extend unbroken from a transducer 140 to a directional cell or to the enter cell. However, it is preferred that a manifold 130 be provided to enable connections of tubes 125 inside and outside of the operational box 150. The manifold 130 may include a plurality of prongs 132. In one aspect, each of the prongs 132 extends from the wall 152 of the operational box 150 and defines a channel that extends from each side of the manifold 130. This means that each prong 132 is actually a pair of prongs, with one prong of a pair of prongs extending inside of the operational box 150, and another prong of the pair of prongs extending outside of the operational box 150. In this way, each pair of prongs 132 enables fluid communication through the tubes 125 without necessity of the operator opening the box and exposing the delicate transducers 140. Further, the therapist or other operator is not required to manipulate the fragile connection 142 between the tubes 125 and the respective transducers 140. Preferably, the tubes 125 are color-coded with the prongs 132 so that the tubes 125 properly correspond to the correct transducers 140. Alternatively, other coding systems may be used such as alphabetical or numeric associations, or the use of symbols. Alternatively still, custom connectors which connect the tubes 125 to the prongs 132 in only one orientation may be utilized.

It is noted again that the tubes 125 are preferably bundled by a tubular sheath 120. The tubular sheath 120 extends generally from proximate the manifold 130 to proximate the manifold 340 of the mouth piece 110. A proximal end 122 of the tubular sheath 120 begins near the manifold 130 of the operational box 150, while a distal end 126 of the tubular sheath 120 covers an end opening towards the mouth piece 110. In this way, the mouth piece 110, the tubes 125 outside of the operational box 150, and the tubular sheath 120 are essentially one integral unit. Each patient is supplied with his or her own mouth piece 110 having integrated tubes 125 and the tubular sheath 120. The only “assembly” required by the therapist or care giver is to connect the mouth piece 110 and tubes 125 with the external prongs 132 on the manifold 130.

In the arrangement of FIG. 1, the transducers 140 are shown external to the head set 200, meaning they are not mechanically supported by the head set 200. However, in an alternate arrangement the transducers 140 are integrated into the head set 200. In the arrangement of FIG. 2A, transducers 240 may be placed inside of the cushioning member 215. Dashed lines are shown in FIG. 2A to indicate the optional placement of transducers 240 on the head set 200A. In this instance, the circuit board 144 will also reside within or along the cushioning member 215.

In any arrangement, the transducers 140/240 are designed to convert changes in pressure within the cells “L,” “R,” “U,” “D,” “E” to electrical signals. The electrical signals may be analog voltage signals. Other examples of electrical signals that may be used include current signals or resistive changes. The changes in pressure within the cells “L,” “R,” “U,” “D,” “E” are delivered pneumatically or fluidically, depending on the fluid used, to the transducers 140/240 through the respective tubes 125. As the transducers 140 sense an increase in pressure, a corresponding voltage or other electrical signal is delivered through the electrical circuit board 144.

The intra-oral system 100 also includes a processor 145, or micro-controller. The processor 145 uses operational software for processing the electrical signals. As shown in the arrangement for the system 100 of FIG. 1, the electrical signals are delivered to the processor 145 by means of the electrical circuit board 144. This means that the processor 145 also resides within the operational box 150. However, in another embodiment the processor 145 resides outside of the operational box 150. In yet another arrangement, electrical signals may be sent through a wireless connection such as through the use of Bluetooth technology. In yet another arrangement, the processor 145 may reside on the head set 200 itself. Such an alternative arrangement is shown in FIG. 2C.

FIG. 2C is a perspective view of a head set 200C, in an alternative arrangement. Here, the transducers 140 and the processor 145 are placed onto the head set 200C itself. The processor 145 may be, for example, an Atmel® AVR® 8-bit micro-controller, useful for C and assembly programming. As another example, the processor 145 may be the Atmel® 8-bit AVR RISC-based micro-controller that combines 16 KB ISP flash memory, 1 KB SRAM, 512B EEPROM, and an 8-channel/10-bit A/D converter (TQFP and QFN/MLF). The device supports a throughput of 20 MIPS at 20 MHz and operates between 2.7 and 5.5 volts. The head set 200C also includes a power switch 255. Jumper tubes 245 provide fluid communication between fluid tubes 125 and the respective transducers 140.

Of interest, the head set 200C may be used for a wireless transmission of signals to a personal digital assistant such as an iPad®. In FIG. 2C, the head set 200C includes a transmitter 160. The transmitter 160 communicates with a second processor using an infrared controller. Alternatively, the transmitter 160 is a transceiver that communicates with a second processor using an RF signal, or by using other wireless means such as Bluetooth, Wi-Fi, Zigby, or Wi-Max.

The head set 200C is intended to be free-standing. In this respect, the fluid tubes 125 connect directly from the mouth piece 110 to the head set 200C without being connected to a separate computer (such as operational box 150 of FIG. 1). Optionally, an opening 233 is provided in the first arm portion 232 or the pivot point 235 for receiving the tubular sheath 120. The second arm portion 236 may then be tubular, so as to guide the tubular sheath 120 to the mouth piece 110.

It is preferred that the wireless head set 200C be powered through a battery. Where a battery is not used, an electrical cord 250 extends from the head set 200C. The cord 250 connects to a power pack (not shown), that may then plug into an electrical outlet for power. Alternatively, the electrical cord 250 has a USB connector (not shown) for placing the processor 145 and other electronics in electrical communication with a computer, such as a lap top (not shown), for power feed.

In any instance, the electrical signals, such as voltage signals, from the transducers 140 are modulated to generate pressure readings from the cells “L,” “R,” “U,” “D,” “E”. If a transducer senses a pressure change of a certain amount from cell “L,”, a cursor (shown at 605 in FIGS. 6A, 6B and 6C) will move one incremental place to the left on a screen (shown at 600A, 600B and 600C). If a transducer senses a pressure change of a certain amount from cell “R,” the cursor will then move one incremental place to the right. Up “U” and down “D” cells are also provided for manipulating the cursor in up and down directions. Additionally, an enter “E” cell may be pressed to activate a function.

Of interest, the cells “L,” “R,” “U,” “D,” “E” are spaced-apart on the under-surface 332 of the palate member 330. This allows for precision in sensing pressure applied by the user's tongue. Thus, the processor 145 need not construct a complex pressure profile to determine where to send the cursor as it is simply reading left, right, up and down signals in an x-y system.

Referring again to FIG. 1, an electrical cord 156 extends from the operational box 150. The cord 156 extends from an opening 157 in the operational box 150. The cord 156 preferably has a USB connector 158 for placing the processor 145 in electrical communication with a computer 160. More specifically, the USB connector 158 places the processor 145 in electrical communication with a processing unit 162 for a computer 160.

The computer 160 is preferably a general purpose computer 160. Such a computer may be a laptop computer or a desk top computer as may be purchased at a local retail store or on-line from an electronics store. In this instance, communications software may be loaded onto the processing unit 162 by the therapist or IT representative or field representative. However, the processing unit 162 may be a specially designed or dedicated unit that comes with the operational box 150. Alternatively, the processing unit 162 may be a central processing unit that is part of a network.

In operation, the system 100 preferably allows a patient to manipulate a cursor on a screen. This is done by the patient moving his or her tongue against the under-surface 332 of the mouth piece 300. Such movement causes an increase in pressure within selected cells. The increase in pressure causes a corresponding increase in pressure within the tubes 125. The pressure changes, in turn, are transmitted to the respective transducers 140 within the operational box 150.

Electrical signals are generated by the transducers 140 in response to the changes in pressure within the tubes 125. These signals are sent to the processor 145. The processor 145, in turn, modulates the signals and sends them to display software residing on the processing unit 162. Using the display, a cursor (or other object not shown in FIG. 1) is caused to be moved across a display 166. Manipulation of the cursor allows the user to move or to actuate a separate object external to the system 100, or to cause an external action.

To implement this function, the system 100 also includes the visual display 166. The display 166 represents a screen for visualizing the cursor as it is moved by the user. The display 166 may include a stand 168 for supporting the display 166. Preferably, the display 166 is adjustable to accommodate the height or position of the user. A cord 165 is offered to provide the needed electrical communication between the graphics processing unit 162 and the display 166 when the two are not part of an integral device such as a laptop computer.

It is understood that the display 166 arrangement of FIG. 1 is merely illustrative. The display 166 may be part of a laptop computer. Alternatively, the display 166 may be part of a headset, or may comprise a large, wall-mounted screen. Alternatively still, the display 166 may be a screen that receives an image from a projector. Alternatively still, the display may be a tablet or so-called smart phone.

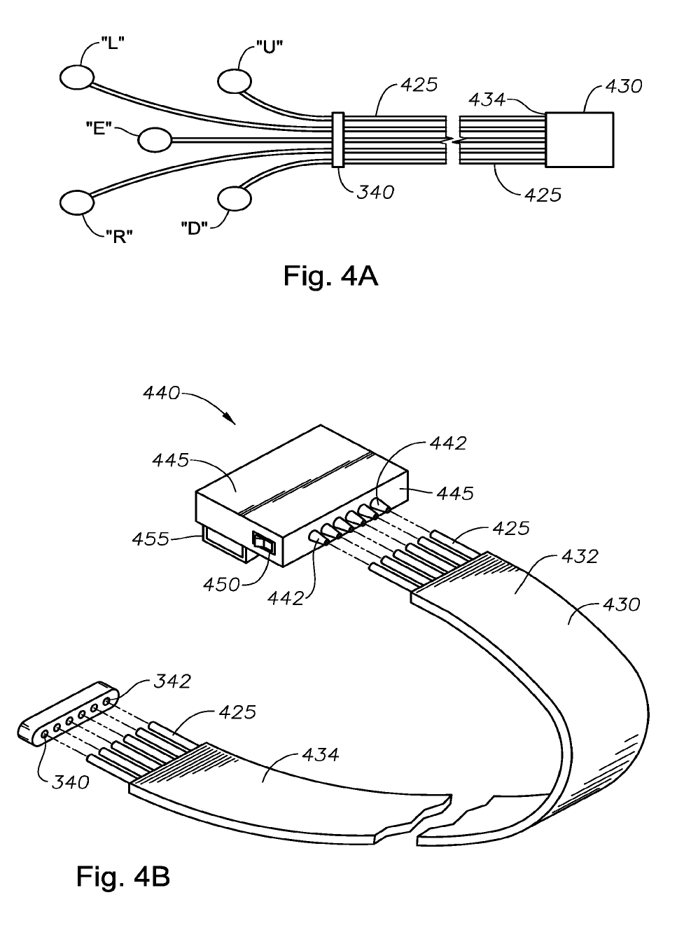

In the arrangement of FIGS. 1, 2A and 2B, the individual tubes 125 are bundled into a circular sheath 120. As an alternative, the intra-oral system 100 may use a ribbon cable. FIGS. 4A and 4B demonstrate the use of a ribbon cable 430.

First, FIG. 4A provides a schematic view of five fluid tubes 425 as may be used in the intra-oral system 100 of FIG. 1, or with head set configuration 200C. The individual tubes 425 extend through the manifold 340 and to the respective fluid cells “L,” “R,” “U,” “D,” and “E.” The tubes 425 extend from the distal end 434 of a ribbon cable 430. The ribbon cable 430 secures the tubes 425 for safety and for ease of use.

FIG. 4B is a perspective view of the ribbon cable 430 of FIG. 4A. Here, proximal 432 and distal 434 ends of the ribbon cable 430 are shown. Tubes 425 extend from each end 432, 434 of the cable 430. In the arrangement of FIG. 4B, the proximal end 432 connects to an electronics box 440, while the distal end 434 connects to the manifold 340 of the mouth piece 300. The connections may be made through a simple friction fit. At the proximal end 432, the tubes 425 slide either into or over nozzles 442 that extend from the electronics box 440. At the distal end 434, the tubes 350 are received into the through-openings 342. Preferably, the ribbon cable 430 is at least about six inches in length.

The electronics box 440 is configured to house electronics for operating the intra-oral system of FIG. 1. The electronics may include transducers, a processor, a battery, and an integrated circuit board. Preferably, the processor will be consolidated with memory and an analog-to-digital converter in one chip. The electronics box 440 may also include short jumper tubes that place the tubes 425 of the ribbon cable, or tube bundle 430 in fluid communication with the transducers via the nozzles 442. These various components are not visible in FIG. 4B, as the electronics box 440 is shown in a closed state. Walls 445 are seen forming the electronics box 440.

FIG. 4B does show a power switch 450 associated with the electronics box 440. The power switch 450 enables the health care provider or assistant to conserve battery power when the electronics box 440 is not being used. Of course, it is understood that the electronics box 440 may alternatively be powered by a power cable (not shown) that may be plugged into an outlet.

The electronics box 440 also includes a data port 455. The data port 455 may be a USB port for connecting the electronics box 440 with a general purpose computer using a cable. The data port 455 may alternatively be a transmitter that transmits signals from the processor to a general purpose computer. In this instance, the transmission is wireless, and communicates with a second processor using an RF signal, or by using other wireless means such as Bluetooth, IR, or Wi-Fi.

The tube bundle 430 arrangement of FIG. 4B is ideal as it allows tubes 425 to be selectively connected and disconnected to a mouth piece 300 and the electronics box 440. This facilitates periodic cleaning of the tubes 425 and the ability of each user or patient to save his or her own tube bundle 430 when the mouth piece 300 is replaced. Of course, it is understood that the tube bundle 430 may alternatively be designed to be integral to the mouth piece 300, the electronics box 440, or both.

In the arrangement of FIG. 4B, the electronics box 440 is ideally designed to reside on a head set, such as head set 200C. However, in another arrangement the electronics box 440 may be integral to a short ribbon cable itself. In this way, a head set is not required; instead, the mouth piece 300 is free-standing with the processor.

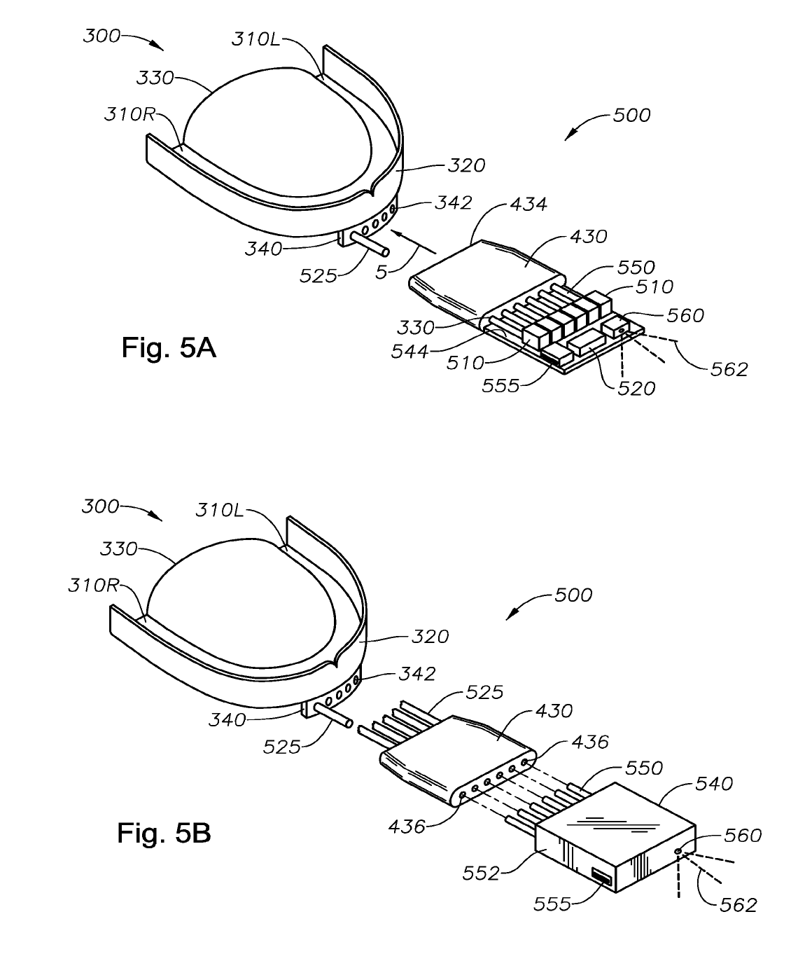

FIGS. 5A and 5B present perspective views of the mouth piece 300 of FIG. 3A in such an arrangement. Here, the mouth piece 300 receives an electronics box 540 that connects immediately to a short ribbon cable 430. Thus, an integrated processing unit 500 is provided.

In FIGS. 5A and 5B, a single illustrative tube 525 is shown extending from a through-opening 342 in the manifold 340. Of course, it is understood that a tube 525 will typically extend from each through-opening 342, and will then be received into an opening (not shown) in the distal end 434 of the ribbon cable 430. This arrangement allows for a quick and easy connection of the mouth piece 300 to a processing unit 500. This further allows for a quick disconnect so that the processing unit 500 may be attached to a different mouth piece 300, either for the same user or for a different user.

It is noted that the processing unit 500 of FIG. 5A does not have a housing. This allows for components of the processing unit 500 in the drawing to be seen. Such components include transducers 510, a controller 520, and a printed circuit board 544. The transducers 510 operate in accordance with transducers 140 shown in FIG. 1 and described above. Similarly, the controller 520 operates in accordance with processor 145 of FIG. 1 described above. In this respect, the controller 520 uses operational software for processing electrical signals from the transducers 510. The electrical signals are delivered to the controller 520 by means of the circuit board 544. The electrical signals, such as voltage signals, are then interpreted to move a cursor on a display.

The processing unit 500 also includes a transmitter 560. This allows a signal to be sent from the processing unit 500 to a second processor via wireless communications network. The second processor is preferably associated with a tablet or personal digital assistant. The tablet or personal digital assistant will include a software program or “app.” This beneficially allows the paraplegic, the quadriplegic, or the severe stroke victim to send text message, send e-mails, navigate web sites, and operate external mechanical and electrical devices through lingual manipulation.

In FIG. 5A, a signal 562 is shown being sent by the transmitter 560. The signals 562 may be transmitted as radio-frequency (RF) or infrared (IR) signals. Alternatively, the signals 562 may be other wireless means such as Bluetooth, Wi-Fi, or Wi-Max.

FIG. 5B offers another view of the mouth piece 300. The ribbon cable 430 and electronics box 540 of FIG. 5A are also seen. Here, a housing 552 is provided for the processing unit 500. The processing unit 500 is shown exploded away from the ribbon cable 430. This exposes jumper tubes 550, which are seen extending away from the electronics box 540. A distal end of each of the tubes 550 is configured to be received in corresponding channels 436 of the ribbon cable 430. Ideally, the rigid fluid tubes 550 will extend at least half an inch to an inch into the channels 436 to provide mechanical support for the electronics box 540.

It is noted that the processing unit 500 also includes a data port 555. In FIG. 5B, the data port 555 is shown as a USB connection. However, it is understood that other types of data ports may be developed and used, presently or in the future. The data port 555 allows for a wired communication between the processing unit 500, as a first processor, and a separate computer as a second processor.

Other components may be used with the processing unit 500 and the electrical circuit board 544. These may include a battery, a clock or timer, resistors, capacitors, and the like.

FIGS. 6A through 6C present various arrangements for displays from the system of FIG. 1. FIGS. 6A through 6C also demonstrate methods for using the head set 200A/200C and attached mouth piece 110.

First, FIG. 6A demonstrates how the head set 200A or 200C and attached mouth piece 110 may be used for moving a mechanical object, in one embodiment. FIG. 6A specifically shows a display 600A. In this system, a cursor is schematically shown at 605. The cursor 605 is used to move an object by the user through lingual manipulation using the directional cells. As the user applies pressure to the directional cells in the mouth piece 110, the cursor 605 is moved across the display 600A. Thus, the mouth piece 110 becomes a “mouth mouse.”

The display 600A is arranged for the purpose of allowing the user to move an external object. In this instance, the individual may use the system 100 to operate a wheelchair. Alternatively, the individual may use the system 100 to manipulate the position of a bed or to open or close a door.

The display 600A includes directional keys. In this arrangement, the directional keys are used to move a wheelchair (not shown). The illustrative directional keys represent forward 610F and reverse 610R arrows. Actuation of these arrows 610F, 610R using the “enter” cell on the mouth piece 300 causes the wheelchair (or other object) to move forward or backward. The directional keys also represent clockwise 615F and counter-clockwise 615R arrows. Actuation of these arrows 615F, 615R causes the wheelchair to rotate clockwise or counter-clockwise.

The keys 610F, 610R, 615F, 615R are activated by using the cursor 605. In one aspect, a symbol 610F, 610R, 615F, or 615R is activated by the user positioning the cursor 605 over the selected symbol 610F, 610R, 615F, 615R, and then either single-clicking or double-clicking on the center (or “enter”) cell “E.” In another aspect, a symbol 610F, 610R, 615F, or 615R is activated by the user positioning the cursor 605 over the selected symbol 610F, 610R, 615F, 615R, and then pressing against the enter cell “E” for a designated period of time at a certain level of pressure.

The display 600A of FIG. 6A is ideally supported on the individual's wheelchair. For example, the display 600A will be mounted on an arm rest (not shown). At the same time, the mouth piece 110 is part of the head set 200A/200C so that the mouth piece 110 is at all times in proximity to the user's mouth. Alternatively, the mouth piece is connected to an integrated processing unit such as unit 500 shown in FIGS. 5A and 5B. In either instance, the individual may selectively insert the mouth piece 110/300 into their mouth for movement of the wheelchair (or other object). In addition, the operational box 150 for the transducers 140 and the processor 145, along with the screen 166, are positioned together on the wheelchair or on the bed or even on the head set 200C, depending on the arrangement.

It is understood in this application that the display 166 will be in electrical communication with a motor or servo-system on the wheelchair. In this way, the user's instructions delivered by moving the cursor 605 on the screen 600A cause the wheelchair to respond. Of course, the display 600A may be used to control mechanical objects other than a wheelchair. For example, symbols 610F, 610R, 615F, 615R may be used to move a bed, open and close a door, and the like.

As an alternative, the user may use the head set 200A/200C and connected mouth piece 110 to manipulate a mechanical object without need of a display 600A. For example, simple pressing of a cell in the mouth piece 110 at a designated pressure and/or for a designated period of time may automatically cause the wheelchair to move, or cause a door to be opened, turn on a light, or control another object. Thus, “moving” of an object herein encompasses moving the object with or without a symbol on a display. The object may be a cursor itself, or may be an external object.

The system 100 may be used by a physically-limited individual to operate other apparatus' besides a mechanical object. Such apparatus' may include electrical appliances such as a television, a light fixture, or a thermostat.

FIG. 6B presents a display 600B for the system 100, in an alternate embodiment. A cursor is again shown at 605. The cursor 605 is used to change the status of an electrical appliance by the user through lingual manipulation of cells attached to the under-surface 332 of a mouth piece 300. The display 600B shows arrow 622 for turning on a television, and arrow 624 for turning off a television. The display 600B also shows carrots 627 for adjusting the volume of the television, and carrots 629 for changing the channel.

The display 600B also shows bar 640(1) for turning a first light fixture on and off, and bar 640(2) for turning a second light fixture on and off. The bar configuration 640(1) and 640(2) may also serve a rheostat, thereby adjusting the brightness of a light fixture. The display 600B also shows arrow 632 for opening a door, and arrow 634 for closing the door. This would be done through a servo-motor.

It is understood that displays 600A and 600B are merely illustrative. Other objects and appliances may be controlled through the use of a cursor and symbols. The user may then press or double-click on the enter cell “E” of the mouth piece 300 to turn an object on or off or to adjust its status. A signal is then sent from the system 100 to the electrical apparatus. This signal is preferably a wireless signal such as through infrared technology, Bluetooth technology or other wireless technology that may be known to those of ordinary skill in the art. Additionally, icons other than arrows, or simply words, may be used on the display 600B.

FIG. 6C presents a display 600C for the intra-oral system 100, in yet an alternate embodiment. In this display 600C, a cursor is again shown at 605. The illustrative cursor 605 is an arrow. The cursor 605 is moved across the display 600C in accordance with pressure applied by the user to directional cells “L,” “R,” “U,” and “D.”. In this embodiment, the display 600C includes a keyboard 650. The keyboard 650 and other symbols in the display 600C are used to allow the individual to type text messages using just his or her lingual musculature.

The display 600C includes symbols 652. These symbols 652 may be used, for example, to open and close a door (not shown) or to select an appliance to be controlled. In the arrangement of FIG. 6C, the symbols 652 are shown schematically; however, it is understood that the symbols 652 will preferably show a door or a thermostat or other external device that is to be adjusted. Arrow keys 654, 656 are also provided on the display 600C. Once a device is selected, the user may manipulate the electrical apparatus or mechanical appliance by double-clicking on an arrow key 654, 656. For example, a light fixture may be brightened or dimmed by single-clicking or double-clicking on the arrow keys 654, 654 using the enter cell “E.” Alternatively, the channel of a television or radio may be changed by clicking on the arrow keys 654, 656. Separate arrow keys (not shown) may be used to then adjust the volume.

A signal is sent from the system 100 to the electrical apparatus. This signal is preferably a wireless signal such as through infrared technology, Bluetooth technology or other wireless technology that may be known to those of ordinary skill in the art.

The keyboard 650 allows the physically-limited individual to type in a text message such as an e-mail message to another individual. The individual uses the cursor 605 to select alpha-numeric keys to be “pressed.” Pressing means clicking or otherwise applying pressure to a selected fluid cell in the mouth piece 300. By selecting and “pressing” a series of digital keys on the keyboard 650, a message may be composed. The message may be seen on a visualization screen 658 on the display 600C. The message may then be “sent” by pressing a return arrow 655. In this arrangement, the processor has a wired or wireless Internet connection for delivering the message through a communications network.

The systems and methods of the intra-oral system 100 described herein contemplate transmission of information signals among headset components, sensor/transducer components, and/or processing components. Communication paths couple the components and include any medium for communicating or transferring files and/or data among the components. The communication paths include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths include removable fixed mediums like flash RAM, Universal Serial Bus (USB) connections, RS-232 connections, telephone lines and buses.

As already described above, the intra-oral system 100 includes a processor 145, or micro-controller under an embodiment. The processor 145 receives analog electrical signals from transducers 140 coupled to pressure sensing fluid cells “L,” “R,” “U,” “D,” “E”. The processor 145 may be external to or integrated within a headset 200 worn by a user of the intra-oral system 100. In either case, the processor 145 in cooperation with an analog to digital convertor converts analog signals to digital signals which may then be transmitted in data packets through packet switched networks. Under one embodiment, the processor 145 is communicatively coupled via a wired or wireless communication path to an on-premise LAN of the user. The LAN is further coupled to a WAN thereby providing a communication path from the processor 145 of the intra-oral system 100 to a remote server. It is understood that a residential gateway may allow the connection of a local area network (LAN) to a wide area network (WAN). The WAN can be a larger computer network (such as a municipal WAN that provides connectivity to residences within the municipality), or the Internet. WAN connectivity may be provided through DSL, cable modem, a broadband mobile phone network, or other connections. Applications running on a processor of a residential gateway may execute applications of an intra-oral system 100 that transmit information signals received from an intra-oral system headset 200 (or associated processor 145) to a remote server using a particular protocol. Information signals may be used to monitor activities of a user of the intra-oral system 100.

Under an embodiment, a remote server may store configuration data of the intra-oral system 100. As one example, a user initiates an action through lingual manipulation of a fluid cell. A processor 145 of the intra-oral system 100 forwards packetized data of the action (indicating the particular fluid cell actuated by user) to a remote server through an on-premise LAN. Configuration data of the intra-oral system 100 may include mappings which associate device specific actions or commands with each fluid cell. Such actions or commands may be movement of a cursor on a computer screen or activating a particular light switch. Further, the configuration data may also store local device address information assigned by the LAN and used by the residential gateway and/or LAN to route data to and from such devices.

The remotely stored configuration data may include a mapping which associates fluid cells with an action in view of a target device. When the remote server receives an information signal, i.e. packetized data indicating activation of a fluid cell, one or more applications running on the remote server map the actuated fluid cell to a particular action or command with respect to a particular device. The remote server then generates a command signal and sends it back to the LAN of the user. The command signal includes information of the command along with the LAN address associated with the targeted device. The command data may be expressed in binary form and transmitted in one or more data packets suitable for transmission over packet switched networks.

A residential gateway of the LAN may receive the command signal and route the command signal according to the corresponding addressing information to a particular device. As described above, the particular device may be a computer coupled to the LAN and the command may be movement of a cursor. Alternatively, the device coupled to the LAN may be a thermostat and the command may be a change in temperature. Alternatively, the device coupled to the LAN may be a door (including a pet door), and the command may be a change in position of the door.

Under this embodiment, remote server configuration data may be used to map fluid cells of the intra-oral system 100 to various functional profiles. As already described above, the various fluid cells may be mapped to directional actions for moving a cursor on a display. Under an alternative embodiment, the various fluid cells may be mapped to actions controlling devices/systems in a home including lights, heating/cooling systems, alarm systems, appliances, etc. Each functional profile associates an activity/command and corresponding device with a fluid cell. A functional profile may associate all such cells of the intra-oral system 100 with a particular device or may distribute cell functionality across multiple devices.

The intra-oral system 100 may under an embodiment provide a web interface that may be used to create and manage functional profiles. A third party, health care provider or provider of intra-oral system 100 services may create the functional profiles using the web interface. Alternatively, a user may access the intra-oral system web interface directly to configure fluid cell associations and fluid cell functional profiles. Under this embodiment, the intra-oral system 100 may provide a default functional profile of the fluid cells allowing interaction with the intra-oral system web-interface in a default mode using a dedicated on-premise computer or display device. Accordingly, the user may configure and select among functional profiles to increase the functionality of the intra-oral system 100.

Aspects of the intra-oral system 100 and corresponding methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the systems and methods include: microcontrollers with memory (such as electronically erasable programmable read only memory (EEPROM)), embedded microprocessors, firmware, software, etc. Furthermore, aspects of the systems and methods may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that any system, method, and/or other components disclosed herein may be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, HTTPs, FTP, SMTP, WAP, etc.). When received within a computer system via one or more computer-readable media, such data and/or instruction-based expressions of the above described components may be processed by a processing entity (e.g., one or more processors) within the computer system in conjunction with execution of one or more other computer programs.

From the above description, it can be seen that a method for moving a cursor on a display using lingual manipulation is provided. In one aspect, the method includes providing an intra-oral system for a user. The system includes an elastomeric mouth piece that is dimensioned to reside around the upper teeth of a user. The mouth piece has opposing left and right sides, and an arcuate portion intermediate the left and right sides to form a generally horseshoe-shaped member.

The system also includes a palate member. The palate member resides between and is supported by the left and right sides of the mouth piece.

The system also includes at least two, and preferably five distinct fluid-containing cells. The cells are affixed to an under-surface of the palate member. The cells are configured to respond to pressure applied by the tongue of the user.

The system further includes a plurality of fluid tubes. Each tube has a proximal end and a distal end. The distal end of each of the tubes is in substantially sealed fluid communication with a corresponding cell.

The system additionally includes a plurality of transducers. The transducers reside external to the mouth piece. Each transducer is configured to convert changes in pressure within a corresponding cell to electrical signals. Preferably, each of the plurality of transducers is a pressure sensor having a diaphragm that is sensitive to changes in pressure within a corresponding tube.

The system also includes a processor for processing the electrical signals. The electrical signals are modulated to move the cursor in response to lingual pressure applied to the cells.

The method also comprises placing the plurality of tubes in fluid communication with the corresponding plurality of transducers. This may be done, for example, by connecting the tubes to an intermediate manifold having fluid channels.

The method further includes applying pressure to selected fluid-containing cells. This is done through the tongue of the user. The result is that the cursor is moved across the display.

In one aspect, the mouth piece and connected palate member are affixed to a head set. The head set is configured to be worn on a head of the user. The method then further comprises placing the head set onto the head of the user, and inserting the mouth piece into the user's mouth. Preferably, the processor and the transducers are mechanically supported by the head set. Alternatively, the transducers and the processor are not mechanically supported by the head set but are tethered to the head set through the plurality of tubes.

In one aspect, each cell and each tube contains a compressible fluid. The compressible fluid may comprise air, oxygen, carbon dioxide, nitrogen, or combinations thereof. In another aspect, each cell and each tube contains a non-toxic incompressible fluid. The incompressible fluid may comprise, for example, water. Some combination of the above may also be used.

Preferably, the at least two fluid-containing cells comprise:

a “left” cell for moving the cursor to the left on the display,

a “right” cell for moving the cursor to the right on the display,

an “up” cell for moving the cursor up on the display,

a “down” cell for moving the cursor down on the display, and

an “enter” cell for actuating a symbol on the display.

Movement of the cursor in response to pressure applied to the cells is linear in an x-y plane. Preferably, an application of pressure by the user's tongue on a cell causes the cursor to advance a single incremental unit—left, right, up or down.

In one embodiment, the transducers and the processor are part of a first processing unit. The first processing unit further comprises a plurality of jumper tubes in fluid communication with respective transducers. The intra-oral system further comprises a ribbon cable for receiving respective jumper tubes in channels at a proximal end, and for receiving respective fluid tubes in channels at a distal end. In this way, the fluid cells are placed in fluid communication with the transducers.

The first processing unit preferably further comprises a transmitter. In this instance, the intra-oral system further comprises a second processing unit associated with a display. Here, the transmitter is configured to communicate with the second processing unit via wireless signals to move a cursor on the display.

The tubes are preferably held in a ribbon cable that is at least about 12 inches in length. In this instance, the first processing unit is supported by a head set to be worn on the head of the user. In another embodiment, the first processing unit and the mouth piece are supported together through the fluid tubes, the ribbon cable and the jumper tubes. This forms a free-standing intra-oral system. Here, the ribbon cable is less than about 3 inches in length. In either instance, wireless signals may be sent from the first processing unit to the second processing unit to move the cursor on the associated display.

Moving the cursor on the display is beneficial for a number of possible purposes. In one aspect, the processor is in electrical communication with a motor for moving an external object. The object may be, for example, a bed, a wheelchair, or a door. In another aspect, the processor is in electrical communication with a switch for changing an electrical state of an appliance. The appliance may be, for example, a light fixture, a television, a mechanical door, a pet feeder/waterer, or a thermostat. In these instances, selecting a symbol on the display actuates a mechanical device or an electrical appliance. The symbol may comprise a picture, one or more alphanumeric characters, an arrow, or a geometric figure. In one use, the display comprises a keyboard such that the user may select a series of characters on the keyboard using their tongue to compose a textual message. In one instance, the method further includes selecting a “send” symbol on the display that, when selected by the user, the textual message is sent through a wireless communications system. This is done by moving the cursor to the send symbol using directional cells, and then “clicking” on the send symbol using the enter cell.

A method of providing therapy for intra-oral musculature is also provided herein. The method includes placing a mouth piece at least partially within a patient's mouth. The mouth piece has at least two pressure cells configured to reside within the user's mouth, and an electronics box having a first micro-processor residing outside of the patient's mouth. The method also includes applying pressure to one or more pressure cells of the at least two pressure cells through lingual manipulation.

In response to pressure applied to the pressure cells through lingual manipulation, the method includes converting pressure values into electrical signals. The electrical signals are processed to correlate pressure applied by lingual manipulation on the pressure cells with (i) locations on the mouth piece, (ii) degrees of pressure applied to the pressure cells on the mouth piece, or (iii) both. The method then includes transmitting the electrical signals as wireless signals from the mouth piece to a second micro-processor

The method additionally includes analyzing the wireless signals. The signals are analyzed to determine a patient's (i) lingual strength, (ii) lingual dexterity, or (iii) both as part of a therapeutic treatment for (i) deglutition, (ii) lingual strength, or (iii) both.

Preferably, the mouth piece comprises a pliable palate member dimensioned to be received against a palate of the patient. Each of the at least two cells is located on a surface of the palate member. The mouth piece further comprises two or more transducers associated respectively with the two or more pressure cells. The electronics box then further comprises two or more transducers associated respectively with the two or more pressure cells, and a transmitter in electrical communication with the transducers. Transmitting the electrical signals as wireless signals from the mouth piece comprises transmitting the electrical signals as wireless signals from the transmitter.

In one aspect of the method, the mouth piece is affixed to a head set configured to be worn on a head of the patient. The method then comprises placing the head set onto the head of the patient, and inserting the mouth piece into the patient's mouth.

Preferably, each of the pressure cells is a fluid pressure cell. Each fluid pressure cell is in fluid communication with a fluid tube that extends to a respective transducer in the electronics box. The further comprises inserting the mouth piece into the patient's mouth, and analyzing the wireless signal comprises reviewing recorded signals over time.

The above descriptions are not intended to be limiting of scope of the inventions. While it will be apparent that the inventions herein described are well calculated to achieve the benefits and advantages set forth above, it will be appreciated that the inventions are susceptible to modification, variation and change without departing from the spirit thereof.

I claim:

1. A method of controlling an external device using lingual manipulation of one or more pressure cells, the method comprising:
placing a mouth piece at least partially within a user's mouth, the mouth piece having at least two pressure cells configured to reside within the user's mouth, and an electronics box having a first micro-processor and configured to reside outside of the user's mouth;
applying pressure to one or more pressure cells of the at least two pressure cells through lingual manipulation;
in response to the pressure applied to the at least two pressure cells, converting pressure values into electrical signals;
processing the electrical signals to correlate pressure applied by lingual manipulation on the pressure cells with (i) locations on the mouth piece, (ii) degrees of pressure applied to the pressure cells on the mouth piece, or (iii) both;
transmitting the electrical signals as wireless signals from the mouth piece to a second micro-processor, and
in response to the receiving of wireless signals, the second micro-processor causing an external object to be moved.

2. The method of claim 1, wherein:
the mouth piece is affixed to a head set configured to be worn on a head of the user; and
the method further comprises placing the head set onto the head of the user, and inserting the mouth piece into the user's mouth.

3. The method of claim 2, wherein:
the mouth piece comprises a pliable palate member dimensioned to be received against a palate of the user; and
each of the at least two pressure cells is located on a surface of the palate member.

4. The method of claim 1, wherein:
the mouth piece comprises a pliable palate member dimensioned to be received against a palate of the user; and
each of the at least two cells is located on a surface of the palate member;
the electronics box further comprises two or more transducers associated respectively with the two or more pressure cells, and a transmitter in electrical communication with the transducers; and
transmitting the electrical signals as wireless signals from the mouth piece comprises transmitting the electrical signals as wireless signals from the transmitter.

5. The method of claim 4, wherein the at least two pressure cells comprise:
a “left” directional pressure cell for moving a cursor to the left on a display,
a “right” directional pressure cell for moving a cursor to the right on a display,
an “up” directional pressure cell for moving a cursor up on a display, and
a “down” directional pressure cell for moving a cursor down on a display.

6. The method of claim 4, wherein:
the at least two pressure cells further comprise an “enter” pressure cell for executing a command on a symbol on a display.

7. The method of claim 6, wherein:
the pressure cells are fluid pressure cells;
each fluid pressure cell is in fluid communication with a fluid tube that extends to a respective transducer in the electronics box.

8. The method of claim 7, wherein:
the one or more symbols on the display comprises a picture, one or more alphanumeric characters, an arrow, or a geometric figure; and
selecting one or more symbols comprises moving a cursor by applying lingual pressure to the two or more pressure cells, and then applying lingual pressure to an enter cell once the cursor has reached a desired symbol.

9. The method of claim 8, wherein:
the second micro-processor is in electrical communication with a motor for moving the external object.

10. The method of claim 1, wherein:
the second micro-processor controls a switch for changing a state of an electrical appliance.

11. The method of claim 8, wherein the one or more symbols comprises a virtual keyboard such that the user may select a series of characters on the keyboard using their tongue to compose a textual message.

12. The method of claim 4, wherein:
the user is a patient;
the second micro-processor is in electrical communication with a display for presenting pressure readings from the pressure sensors; and
the method further comprises analyzing the wireless signals to determine a patient's (i) lingual strength, (ii) lingual dexterity, or (iii) both as part of a therapeutic treatment for (i) deglutition, (ii) lingual strength, or (iii) both

13. A method of providing therapy for intra-oral musculature, the method comprising:
placing a mouth piece at least partially within a patient's mouth, the mouth piece having at least two pressure cells configured to reside within the user's mouth, and an electronics box having a first micro-processor and configured to reside outside of the patient's mouth;
applying pressure to one or more pressure cells of the at least two pressure cells through lingual manipulation;
in response to pressure applied to the pressure cells through lingual manipulation, converting pressure values into electrical signals;
processing the electrical signals to correlate pressure applied by lingual manipulation on the pressure cells with (i) locations on the mouth piece, (ii) degrees of pressure applied to the pressure cells on the mouth piece, or (iii) both;
transmitting the electrical signals as wireless signals from the mouth piece to a second micro-processor; and
analyzing the wireless signals to determine a patient's (i) lingual strength, (ii) lingual dexterity, or (iii) both as part of a therapeutic treatment for (i) deglutition, (ii) lingual strength, or (iii) both.

14. The method of claim 13, wherein:
the mouth piece is affixed to a head set configured to be worn on a head of the patient; and
the method further comprises placing the head set onto the head of the patient, and inserting the mouth piece into the patient's mouth.

15. The method of claim 14, wherein:
the mouth piece comprises a pliable palate member dimensioned to be received against a palate of the patient; and
each of the at least two cells is located on a surface of the palate member.

16. The method of claim 13, wherein:
the mouth piece comprises a pliable palate member dimensioned to be received against a palate of the patient;
each of the at least two cells is located on a surface of the palate member;
the mouth piece further comprises two or more transducers associated respectively with the two or more pressure cells;
the electronics box further comprises two or more transducers associated respectively with the two or more pressure cells, and a transmitter in electrical communication with the transducers; and
transmitting the electrical signals as wireless signals from the mouth piece comprises transmitting the electrical signals as wireless signals from the transmitter.

17. The method of claim 16, wherein the at least two pressure cells comprise:
a “left” directional pressure cell for moving a cursor to the left on a display,
a “right” directional pressure cell for moving a cursor to the right on a display,
an “up” directional pressure cell for moving a cursor up on a display, and
a “down” directional pressure cell for moving a cursor down on a display.

18. The method of claim 16, wherein:
the pressure cells are fluid pressure cells; and
each fluid pressure cell is in fluid communication with a fluid tube that extends to a respective transducer in the electronics box.

19. The method of claim 16, wherein:
the method further comprises inserting the mouth piece into the patient's mouth; and
analyzing the wireless signal comprises reviewing recorded signals over time.

20. A method of providing therapy for intra-oral musculature, the method comprising:
placing a mouth piece at least partially within a patients mouth, the mouth piece having at least two fluid pressure cells;
applying pressure to one or more pressure cells of the at least two fluid pressure cells through lingual manipulation by the patient;
in response to pressure applied to the one or more fluid pressure cells, converting pressure values into electrical signals;
processing the electrical signals to correlate pressure applied by lingual manipulation on the pressure cells with (i) locations on the mouth piece, (ii) degrees of pressure applied to the pressure cells on the mouth piece, or (iii) both;
transmitting the electrical signals as wireless signals from the mouth piece to an external micro-processor, and
analyzing the wireless signals to develop a patient's intra-oral musculature as part of a therapeutic treatment for lingual manipulation, chewing and/or swallowing.

21. The method of claim 20, wherein:
the mouth piece comprises a pliable palate member dimensioned to be received against a palate of the patient;
each of the at least two fluid pressure cells is located on a surface of the palate member;
the mouth piece further comprises two or more transducers associated respectively with the two or more pressure cells by means of respective fluid tubes;
the mouth piece further comprises an electronics box residing external to the patient's mouth and housing the two or more transducers and a transmitter in electrical communication with the transducers; and
transmitting the electrical signals as wireless signals from the mouth piece comprises transmitting the electrical signals as wireless signals from the transmitter.
